(19) United States
(12) Patent Application Publication
(10) Pub. No.: US 2009/0118591 A1
(43) Pub. Date: May 7, 2009

(54) METHOD AND APPARATUS FOR COLLECTING DATA FROM HOUSEHOLD MEDICAL DEVICES

(75) Inventors: Minjoon Kim, Daejeon (KR); Jung Kim, Daejeon (KR)

Correspondence Address: LAHIVE & COCKFIELD, LLP;FLOOR 30, SUITE 3000, ONE POST OFFICE SQUARE, BOSTON, MA, 02109, US

(73) Assignee: H3 SYSTEM CO., LTD., Daejeon (KR)

(21) Appl. No.: 11/934,593

(22) Filed: Nov. 2, 2007

(51) Int. Cl. A61B 5/00

(52) U.S. Cl. 600/300

(57) ABSTRACT

Disclosed is a communication protocol for collecting data from household medical devices, the protocol including the steps of: transmitting an initialization packet in order to detect a household medical device capable of communication, and receiving a reply packet including device type/id information from the device; asking the device for a number of stored data items, and receiving a reply; and asking the device for measurement data, and receiving at least one data item from the device, wherein each of the packets for the communication includes a plurality of fields including start code, size, command, device type, device id, and checksum. Accordingly, in data exchange between a Home gateway and a household medical device, a compatible protocol is employed, so that the Home gateway can receive measurement data from various types of medical devices, the protocol can be applied to various manufacturers' products, and the protocol has an extensibility of application even to newly developed medical devices in the future, which enables users to easily select medical devices.

### BACKGROUND OF THE INVENTION

1. Field of the invention

The present invention relates to a communication method and apparatus for collecting data from household medical devices, and more particularly to a communication method and apparatus for collecting data from household medical devices, which employ a compatible protocol in data exchange between a Home gateway and a household medical device, so that the Home gateway can receive measurement data from various types of medical devices, the protocol can be applied to various manufacturers' products, and the protocol has an extensibility of application even to newly developed medical devices in the future, which enables users to easily select medical devices.

2. Description of the Related Art

Conventionally, in collection of various health-related measurements of patients, such as blood sugar, blood pressure, body weight, etc, a healthcare center received handwritten health-related measurements from patients, so that doctors of the healthcare center refer to the measurements in medical treatment. Recently, with rapid spread of the Internet, a method of collecting health-related measurement data online has been introduced.

In order to collect health-related data online, a system provided with a Home gateway is used. In the system, when a patient connects a device which has been used for measurement to the Home gateway, the Home gateway receives data from the device, stores the data therewithin, and transfers the data to a healthcare server via a communication network, thereby enabling doctors of the healthcare center to refer to the data online.

In design of a data exchange protocol between a Home gateway and medical devices, respective medical device manufacturers have not used general-purpose protocols, but have made and used protocols suitable for characteristics of their own products. For example, in a case of a manufacturer manufacturing only a blood sugar level meter and a Home gateway belonging to the blood sugar level meter, since there is no need to differentiate the types of data collection devices, a protocol has been made based on the assumption that a data collection medical device is a blood sugar level meter. Also, in a case of even a manufacturer manufacturing various medical devices, a protocol has been made based on the manufacturer's own medical devices without regard to medical devices made by other manufacturers, so that it is impossible to apply the protocol to medical devices made by other manufacturers. Moreover, although transferred data lengths of respective devices may be different from each other, there is no compatible protocol applicable to such various medical devices.

As described above, when a manufacturer of a Home gateway and a manufacturer of a medical device are different from each other, communication between the medical device and the Home gateway is impossible because of incompatibility of a protocol. Also, if a new medical device is released in the market, application of the new medical device is impossible because of inextensibility of a protocol. Accordingly, a user, who has purchased a Home gateway, has to purchase a medical device made by the same manufacturer of the Home gateway. If the user uses a medical device of which a manufacturer is different from a relevant Home gateway manufacturer, there is a problem in that the user has to inevitably purchase and use a Home gateway made by another medical device manufacturer, or has to give up transferring measurement data via online because of lack of compatibility in terms of communication.

### SUMMARY OF THE INVENTION

Accordingly, the present invention has been made to solve the above-mentioned problems occurring in the prior art, and the present invention provides a communication protocol and apparatus for collecting data from household medical devices, which employ a compatible protocol in data exchange between a Home gateway and a medical device, so that the Home gateway can receive measurement data from various types of medical devices, the protocol can be applied to various manufacturers' products, and the protocol has an extensibility of application even to newly developed medical devices in the future, which enables users to easily select medical devices.

In accordance with an aspect of the present invention, there is provided a communication protocol for collecting data from household medical devices, the protocol including the steps of: transmitting an initialization packet in order to detect a household medical device capable of communication, and receiving a reply packet including device type/id information from the device; asking the device for a number of stored data items, and receiving a reply; and asking the device for measurement data, and receiving at least one data item from the device, wherein each of the packets for the communication includes a plurality of fields including start code, size, command, device type, device id, and checksum.

In accordance with another aspect of the present invention, there is provided an apparatus for collecting data from household medical devices, the apparatus including: a communication means for data communication with a household medical device; and a processor for collecting measurement data while communicating with the household medical device through the communication means, by transmitting an initialization packet in order to detect a household medical device capable of communication, and receiving a reply packet including device type/id information from the device; asking the device for a number of stored data items, and receiving a reply; and asking the device for measurement data, and receiving at least one data item from the device.

### BRIEF DESCRIPTION OF THE DRAWINGS

The above and other objects, features and advantages of the present invention will be more apparent from the following detailed description taken in conjunction with the accompanying drawings, in which:

FIG. 1 is a diagram illustrating a communication protocol which implements a communication method for collecting data from household medical devices according to a first embodiment of the present invention;

FIG. 2 is a diagram illustrating formats of packets transmitted by a communication protocol which implements a communication method for collecting data from household medical devices according to a first embodiment of the present invention;

FIG. 3 is a flowchart illustrating a communication protocol which implements a communication method for collecting data from household medical devices according to a first embodiment of the present invention;

FIG. 4 is a flowchart illustrating a Multi_transfer_flow of a communication protocol which implements a communication method for collecting data from household medical devices according to a first embodiment of the present invention;

FIG. 5 is a diagram illustrating a communication protocol which implements a communication method for collecting data from household medical devices according to a second embodiment of the present invention;

FIG. 6 is a flowchart illustrating a communication protocol which implements a communication method for collecting data from household medical devices according to a second embodiment of the present invention; and

FIG. 7 is a diagram illustrating an apparatus for collecting data from household medical devices according to an embodiment of the present invention.

### DETAILED DESCRIPTION OF THE EXEMPLARY EMBODIMENTS

Hereinafter, an exemplary embodiment of the present invention will be described with reference to the accompanying drawings. In the following description and drawings, the same reference numerals are used to designate the same or similar components, and so repetition of the description on the same or similar components will be omitted.

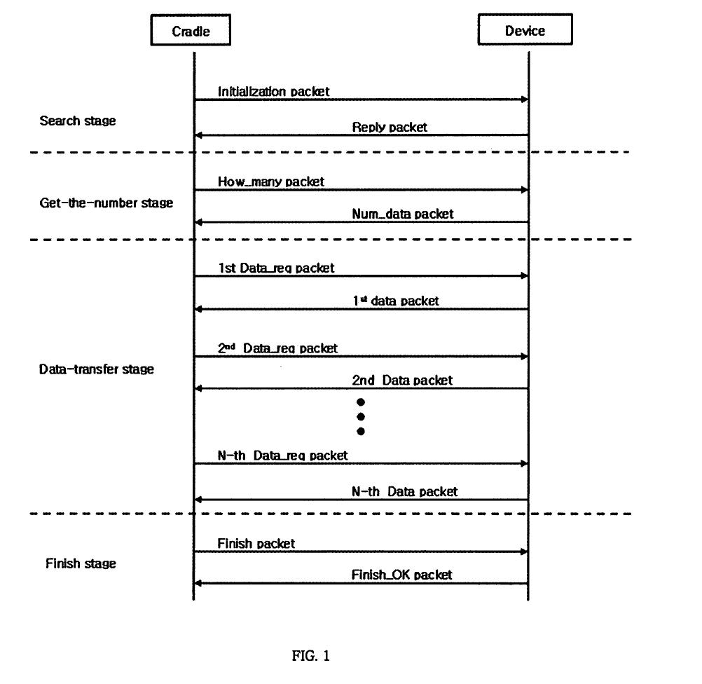

FIG. 1 is a diagram illustrating a communication protocol which implements a communication method for collecting data from household medical devices according to a first embodiment of the present invention.

As shown in FIG. 1, a protocol is largely divided into four stages: search, get-the-number, data-transfer, and finish.

A field structure of a packet used for the protocol basically includes “(start_code) (Size) (Command) (device_type) (device_id) (Data) (Checksum)”.

In a search stage, a Home gateway transmits an initialization packet for detecting a device connected thereto. The device which has received the initialization packet transmits a reply packet having a device type field and a device id field including device type/ID information to the Home gateway. The device type indicates the type of a relevant device, for example, a sphygmomanometer, or a blood sugar level meter, and the device id indicates an ID of a relevant device. The device ID may include a device proper number such as a serial ID, and may indicate additional information on the device type, such as a vendor, a model name, etc.

In a get-the-number stage after the search stage, the Home gateway which has received the reply packet transmits a how-many packet asking how many measurement data items exist in the device. The device which has received the how_many packet transmits a num_data packet having a data field including the number of the measurement data items, to the Home gateway.

After receiving the num_data packet, the Home gateway proceeds to a data-transfer stage, and receives at least one data item if there is data to receive.

When the Home gateway requires only one data item out of measured data, the Home gateway requests the required one data item, and receives a corresponding data packet from the device. On the other hand, when the Home gateway requires two or more data items, the Home gateway transmits a data_req packet and receives a data packet from the device, repetitively, until the number of the required data items is achieved.

After the data-transfer stage, the Home gateway transmits a finish packet indicating the end of data transmission/reception to the device. The device which has received the finish packet replies to the Home gateway by transmitting a finish_OK packet, and then the entire communication process is completed.

In the Search stage of the protocol, there are two modes: a wired mode for communication with a device connected to a wired communication port, and a wireless mode for communication with a wireless communication unit. In the wired mode, a Home gateway transmits an “Are You There”-type initialization packet including type/id information on each device via a time division multiplex (TDM) system. On the other hand, in the wireless mode, a Home gateway transmits a “Who Are You”-type initialization packet asking whether any device is connected through all channels of a wireless communication unit, via a frequency division multiplex (FDM) system.

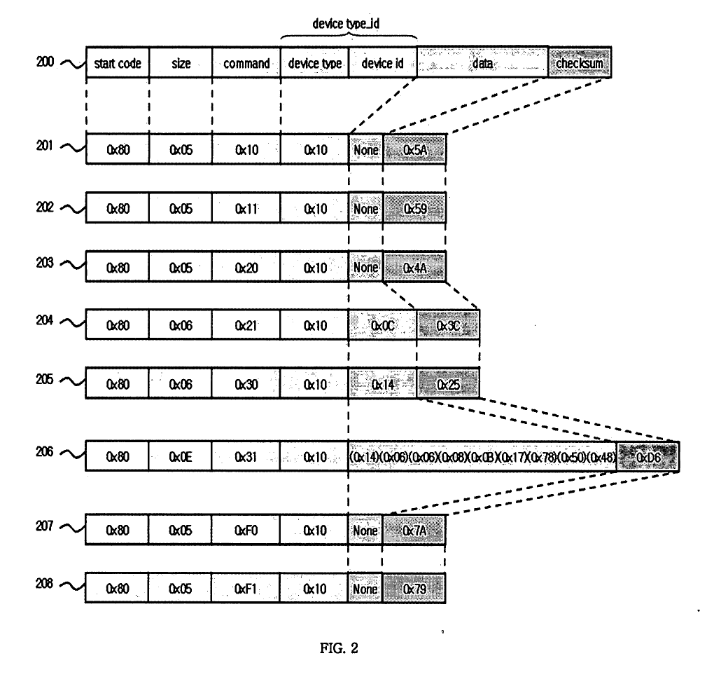

FIG. 2 is a diagram illustrating formats of packets transmitted by a communication protocol which implements a communication method for collecting data from household medical devices according to a first embodiment of the present invention. “None” marked data fields of the packets shown in FIG. 2 indicate 0 byte data. That is, there is no data field. Also, dotted lines between the packets do not indicate that the fields have the same sizes as each other, but indicate that the fields have the same properties as each other.

FIG. 2 illustrates the format of step-by-step transmitted/received packets while a Home gateway collects blood pressure data from a sphygmomanometer connected to a wired communication port. Herein, a protocol including a search stage in a wired mode is used. The value of a checksum field is set in such a manner that the sum total of respective packet values is a specific value, for example, 0xFF. If overflow occurs in the sum total of respective packet values, the value of the checksum field is set in such a manner that the value excluding the overflow is a specific value, for example, 0xFF. Also, total 1 byte is assigned to a device type field and a device id field, and herein higher 4 bit is assigned to the device type field, and lower 4 bit is assigned to the device id field, respectively. For convenience of explanation, a 1 byte field assigned to the device type field and the device id field is called “device_type_id.”

In a search stage, in order to search for a device in which device_type: 1, and device_id: 0, an initialization packet 201 including (start_code: 0x80), (size: 0x05), (command: 0x10), (device_type_id: 0x10), and (checksum) is transmitted to devices. Herein, device_type_id: 0x10 indicates that device_type: 1 and device_id: 0. A device including device_type_id: 0x10, out of the devices which have received the initialization packet 201, transmits a reply packet 202 including (start_code: 0x80), (size: 0x05), (command: 0x11), (device_type_id: 0x10), and (checksum) to a Home gateway, thereby informing of the completion of connection.

The Home gateway which has received the reply packet transmits a how_many packet 203 including (start_code: 0x80), (size: 0x05), (command: 0x20), (device_type_id: 0x10), and (checksum) to the device in order to ask how many measurement data items exist. The device which has received the how_many packet 203 transmits a num_data packet 204 including (start_code: 0x80), (size: 0x06), (command: 0x21), (device_type_id: 0x10), (Data=number_of_data: 0x0C) and (checksum) to the Home gateway, thereby informing of the number of data items. Herein, a data field indicates number_of_data.

After receiving the num_data packet 204, the Home gateway performs a process of receiving data packets equal to number₁₃ of_data from the device. When the Home gateway transmits a data_req packet 205 including (start_code: 0x80), (size: 0x06), (command: 0x30), (device_type_id: 0x10), (Data=pos_data: 0x14) and (checksum) to the device in order to receive the 20^{th} measurement data, the device which has received the data_req packet 205 transmits a data packet 206 including (start_code: 0x80), (size: 0x0E), (command: 0x31), (device_type_id: 0x10), (Data=pos_data+measurement_data: 0x14 0x06 0x06 0x08 0x0B 0x17 0x78 0x50 0x48) and (checksum) to the Home gateway. Herein, the first byte of a data field indicates the sequence (pos_data, that is, a position) of stored measurement data, and thus ‘0x14’ indicates the 20^{th} measurement data. On the other hand, the rest of the data field is measurement_data, and herein “0x06 0x06 0x08 0x0B 0x17 0x78 0x50 0x48” means “measurement date and time: JUN/08/2006 11:23, systolic/diastolic blood pressure: 120/80, and pulse rate: 72”

When all of the measurement data is completely received, or when there is no need to receive the measurement data any longer, the Home gateway transmits a finish packet 207 including (start_code: 0x80), (size: 0x05), (command: 0xF0), (device_type_id: 0x10), and (checksum) to the device, thereby notifying that there is no more data to receive. The device which has received the finish packet 207 transmits a finish_OK packet 208 including (start_code: 0x80), (size: 0x05), (command: 0xF1), (device_type_id: 0x10), and (checksum) to the Home gateway.

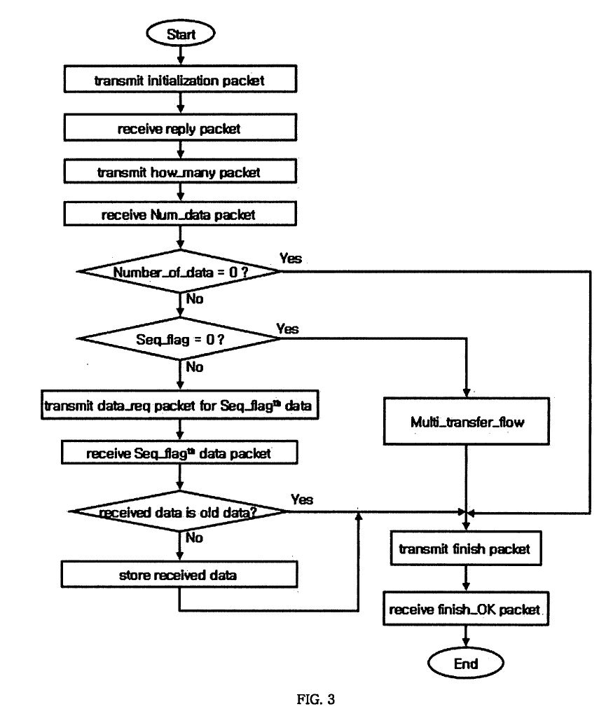

FIG. 3 is a flowchart illustrating a communication protocol which implements a communication method for collecting data from household medical devices according to a first embodiment of the present invention.

FIG. 3 is an example of a wired communication port where a device is connected via a TDM system. When the connection of a device is checked, an initialization packet is transmitted to the device through a relevant communication port. Herein, the initialization packet includes a device type field and a device id field carrying device type/id information, and asks if the device is capable of communication. When a reply packet is received from the device, a how_many packet is transmitted in order to know the number of measurement data items. The how_many packet includes a device type field and a device id field carrying type/id information on the device to receive, and herein, a command field includes a command for transmitting the number of measurement data items.

When a num_data packet is received from the device which has received the how_many packet, a process of receiving measurement data packets is performed. A data field of the received num_data packet includes the number of data items (number₁₃ of_data).

It is checked if number₁₃ of_data is 0. If the number₁₃ of_data is 0, the process proceeds to a finish stage because there is no measurement data. On the other hand, if the number₁₃ of_data is not 0, it is checked if Seq_flag is 0.

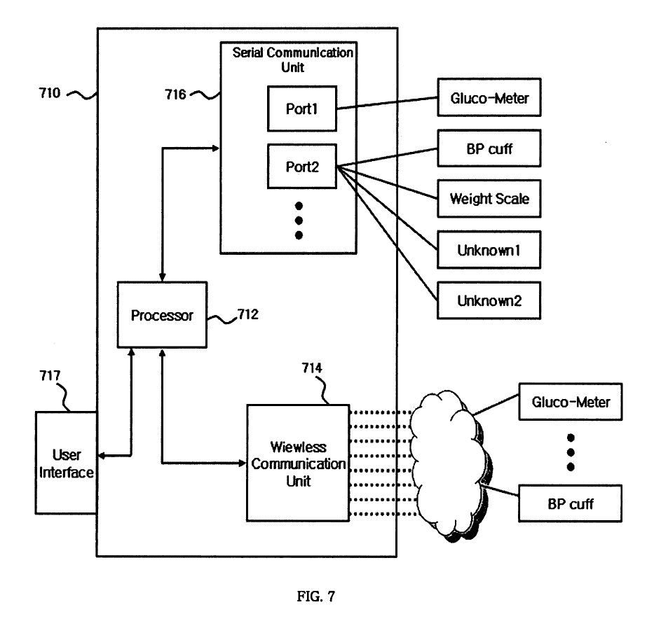

The Seq_flag is a flag where the user sets the sequence of measurement data to receive. If the user wants to receive the second measurement data, the Seq_flag is set to 2, and if the user wants to receive all of the measurement data regardless of the sequence, the Seq_flag is set to 0. This will be described again, with reference to an apparatus for collecting data from household medical devices of FIG. 7.

If the Seq_flag is 0, the process proceeds to Multi_transfer_flow, and then all of the measured data is sequentially transferred. After the data transfer is completed, the process proceeds to the finish stage.

If the Seq_flag is not 0, the device is requested to transfer the Seq-flag^{th} data. When a data packet including the Seq-flag^{th} data is transferred from the device, it is checked if the data stored in pos_data within the data packet is old data. In other words, it is checked if the same data as the received data has been previously stored. As soon as the data is identified as old data, the process proceeds to the finish stage. On the other hand, if the received measurement data is not old data, the measurement data is stored and the process proceeds to the finish stage. However, the old-data identification is not a necessary flow, and thus it is possible to overwrite overlapped data without the identification process.

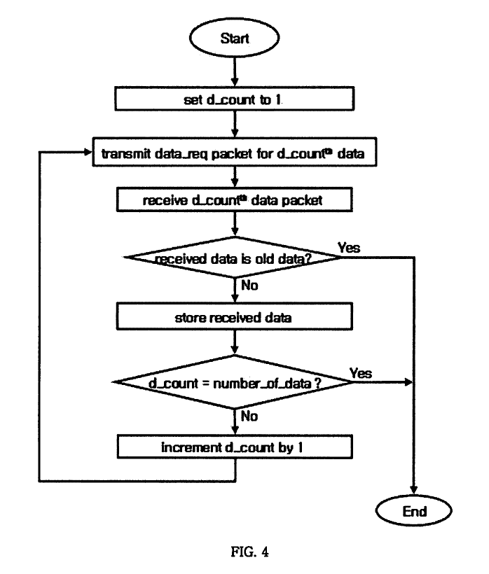

FIG. 4 is a flowchart illustrating a Multi_transfer_flow of a communication protocol which implements a communication method for collecting data from household medical devices according to a first embodiment of the present invention.

According to the Multi_transfer_flow shown in FIG. 4, firstly, d_count is set to 1, and then, a packet requesting the latest first data is transferred. When a data packet including the latest first data is received, it is checked if the measurement data stored in a data field within the data packet is old data. If the same data as the measurement data has been previously stored, the measurement data is identified as old data. As soon as the measurement data is identified as old data, the Multi_transfer_flow ends. On the other hand, when the received measurement data is not old data, the measurement data is stored, and then it is checked if d_count equals to number₁₃ of_data.

The d_count equal to number₁₃ of_data indicates that all of the measurement data is completely received, and thus the Multi_transfer_flow ends. On the other hand, the d_count not equal to number₁₃ of_data indicates that there is more data to receive, and thus the value of d_count is incremented by one, and a data_req packet requesting the latest second data packet is transmitted to the device.

Such data reception continues until received data is identified as old data, or until all of the measurement data is completely received. Then, the Multi_transfer_flow ends.

After the Multi_transfer_flow shown in FIG. 4, the process returns to the finish stage. In the finish stage, a finish packet is transmitted to the device, and a finish_OK packet is received from the device. Then, the entire reception process is completed.

In the reception of measurement data in the Multi_transfer_flow, it is possible to sequentially receive data from the latest to the oldest as shown in FIG. 4, or else it is possible to sequentially receive data from the oldest to the latest. Also, as previously described above, the old-data identification is not a necessary flow, and thus it is possible to overwrite overlapped data without the identification process.

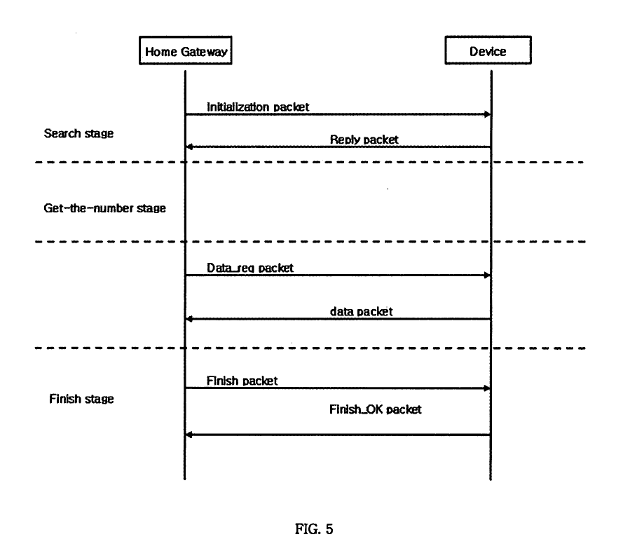

FIG. 5 is a diagram illustrating a communication protocol which implements a communication method which employs a communication protocol for collecting data from household medical devices according to a second embodiment of the present invention.

FIG. 5 is an example of a wireless communication unit where a device is detected via an FDM system. Firstly, an initialization packet is transmitted to respective wireless communication channels. Then, when a reply packet carrying device type/id information is received from a device connected to one of the wireless channels, data exchange with the device starts.

A communication protocol for collecting data from household medical devices according to a second embodiment of the present invention pays main attention to reception of only one data item lately measured by a medical device.

Therefore, in a communication protocol for collecting data from household medical devices according to a second embodiment of the present invention, a get-the-number stage is omitted. Also, in a data-transfer stage, when a data_req packet requesting measurement data transfer is transmitted to a device, the device transmits a data packet storing the measurement data in a data field therewithin to a Home gateway. Then, the Home gateway finishes the data-transfer-stage by receiving the data packet.

After the data-transfer-stage, a finish stage is the same as in the first embodiment.

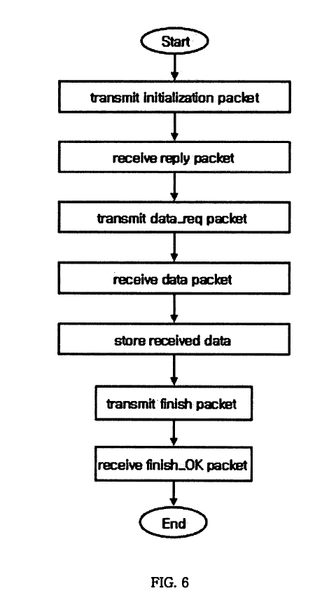

FIG. 6 is a flowchart illustrating a communication protocol which implements a communication method for collecting data from household medical devices according to a second embodiment of the present invention.

In a communication protocol for collecting data from household medical devices according to a second embodiment of the present invention, an initialization packet is transmitted to a device, and a reply packet is received from the device. Then, a Data_req packet is transmitted to the device, and a data packet is received only once. After the reception of the data packet, the received data is stored, and then a finish stage is performed, thereby finishing the transmission.

FIG. 7 is a diagram illustrating an apparatus for collecting data from household medical devices according to an embodiment of the present invention.

An apparatus for collecting data from household medical devices includes a processor 712, a wireless communication unit 714, a serial communication unit 716, and a user interface 717. In the apparatus, the serial communication unit 716 or the wireless communication unit 714 is used for a communication means with an external medical device. In FIG. 7, a wired communication unit in the present invention is realized as the serial communication unit 716. The user selects the sequence of data to receive by pressing a sequence button of the user interface 717. If the user wants to receive all of the measurement data, he/she needs to press a reception-start button of the user interface 717. If the user wants to receive the second data, he/she needs to press ‘2’ of the user interface 717.

Also, a communication protocol for collecting data from household medical devices is used for a communication protocol with an external medical device.

When a medical device is connected to the serial communication unit 716, a Home gateway 710 detects what kind of a medical device is in connection by searching for the device via a TDM system in a search stage of the protocol. After the detection of the device, the number of data items stored in the device is detected in a get-the-number stage of the protocol, and then, the stored data is transferred in a data-transfer stage.

When the transferred data is old data, or when all the data measured by the device is completely received, the process returns to a finish stage, and finishes the entire transmission process.

Although an exemplary embodiment of the present invention has been described for illustrative purposes, those skilled in the art will appreciate that various modifications, additions and substitutions are possible, without departing from the scope and spirit of the invention as disclosed in the accompanying claims.

As described above, in the present invention, a compatible protocol is employed in data exchange between a Home gateway and a household medical device, so that the Home gateway can receive measurement data from various types of medical devices, the protocol can be applied to various manufacturers' products, and the protocol has an extensibility of application even to newly developed medical devices in the future, which enables users to easily select medical devices.

What is claimed is:

1. A communication method for collecting data from household medical devices, the method comprising the steps of:
(a) transmitting an initialization packet in order to detect a household medical device capable of communication, and receiving a reply packet including device type/id information from the device;
(b) asking the device for a number of stored data items, and receiving a reply; and
(c) asking the device for measurement data, and receiving at least one data item from the device,
wherein each of the packets for the communication comprises a plurality of fields including start code, size, command, device type, device id, and checksum.

2. The method as claimed in claim 1, wherein, in step (a), the initialization packet comprises type/id information on a device to detect.

3. The method as claimed in claim 1, wherein, in step (a), the initialization packet comprises command information for requesting type/id information on a device to detect.

4. The method as claimed in claim 1, wherein a total of 1 byte is divided into two parts, which are then assigned to the device type field and the device id field, respectively.

5. An apparatus for collecting data from household medical devices, the apparatus comprising:
a communication means for data communication with a household medical device; and
a processor for collecting measurement data while communicating with the household medical device through the communication means, by transmitting an initialization packet in order to detect a household medical device capable of communication, and receiving a reply packet including device type/id information from the device; asking the device for a number of stored data items, and receiving a reply; and asking the device for measurement data, and receiving at least one data item from the device.

6. The apparatus as claimed in claim 5, further comprising a reception-start button as a user interface.

7. The apparatus as claimed in claim 5, further comprising, as a user interface, a sequence button by which a user selects a sequence of received data.

8. The apparatus as claimed in claim 5, wherein the communication means comprises a wired communication unit or a wireless communication unit.
